(19) United States
(12) Patent Application Publication
(10) Pub. No.: US 2022/0023651 A1
(43) Pub. Date: Jan. 27, 2022

(54) RF TREATMENT DEVICE, MEDICAL RF DEVICE, AND CONTROL METHODS THEREFOR

(71) Applicant: LUTRONIC CORPORATION, Goyang (KR)

(72) Inventors: Kwang Chon KO, Paju (KR); James Bartholomeusz, Beverly Hills, CA (US)

(21) Appl. No.: 17/420,014

(22) PCT Filed: Dec. 9, 2019
(86) PCT No.: PCT/KR2019/017315

§ 371 Date: Jun. 30, 2021

(30) Foreign Application Priority Data
Dec. 31, 2018 (KR) 10-2018-0173463

(51) Int. Cl. A61N 1/40; A61N 1/08

(52) U.S. Cl. CPC A61N 1/403; A61N 2001/083; A61N 1/08

(57) ABSTRACT

The present invention relates to an RF treatment device, a medical RF device, and control methods therefor. Specifically, provided are an RF treatment device, a medical RF device, and control methods therefor, the RF treatment device comprising: an RF generation unit for generating RF energy; a plurality of RF electrodes connected to the RF generation unit through an RF circuit and selectively inserted into body tissue to transmit RF energy to the body tissue; and a sensing unit for sensing a loss of the RF energy transmitted to the body tissue due to the impedance characteristics of the body tissue.

### TECHNICAL FIELD

The disclosure relates to a radio frequency (RF) treatment device, a medical RF device, and control methods thereof, and more particularly to an RF treatment device, a medical RF device and control methods thereof, in which a patient's tissue information is taken into account to carry out optimal treatment

### BACKGROUND ART

A tissue treatment method of using radio frequency (RF) energy may be classified into a contact treatment method where tissue is treated by transferring RF energy to the outer surface of the tissue, and an invasive treatment method where an RF electrode is partially or entirely inserted into tissue to transfer the RF energy. Between them, the invasive treatment method generally employs a needle, a catheter or the like treatment device having a small-diameter insert, and carries out treatment by inserting the treatment device up to a target position inside tissue and then transferring the RF energy to the inside of the tissue.

Such an RF treatment method has been generally used in making an incision, stop bleeding or the like surgical treatment in a lesion of an internal organ. Recently, the RF treatment method has been used for wrinkle removal, scar removal, acne treatment and the like dermal lesion treatment by inserting a needle-type electrode into a skin, and such a technique has also been disclosed in Korean patent publication No. 10-2011-0000790.

The RF treatment method is based on a principle that electric energy flowing through tissue is converted into thermal energy and thus energy is transferred to the tissue when RF current flows in the tissue through an electrode. However, the transferred energy is varied depending the impedance characteristics of the tissue even when RF energy of the same power is transferred. Due to this problem, the transferred energy may not be enough to carry out optimal treatment even when the treatment is carried out under the same condition.

### DISCLOSURE

### Technical Problem

The present invention is to provide a radio frequency (RF) treatment device, a medical RF device, and control methods thereof, in which characteristics of tissue are identified in consideration of impedance characteristics of the tissue in terms of tissue treatment using transfer of RF energy, and appropriate RF energy is transferred based on the identified characteristics of the tissue, thereby having an enough treatment effect.

### Technical Solution

To achieve the object of the disclosure, there is provided a radio frequency (RF) treatment device including: an RF generator configured to generate RF energy; a plurality of RF electrodes connected to the RF generator through an RF circuit, selectively inserted in body tissue, and configured to transfer the RF energy to the body tissue; a sensor configured to detect a loss of RF energy transferred to the body tissue due to impedance characteristics of the body tissue; an impedance adjuster provided on the RF circuit and configured to have variable impedance; and a controller configured to control the impedance adjuster to reduce a loss of RF energy transferred to the body tissue, based on information detected by the sensor.

In this case, the RF electrode may be inserted up to a fat layer in a body and configured to transfer RF energy, and the sensor may be configured to detect a loss of RF energy due to impedance characteristics of the fat layer.

Further, the sensor may be configured to measure power supplied from the RF generator, and voltage and current applied through an RF electrode, and detect a loss of RF energy due to the impedance characteristics of the tissue. Specifically, the sensor may be configured to detect a loss of the RF energy based on power of the RF energy generated by the RF generator and power of RF energy calculated with the measured voltage and current.

Further, the controller may control the impedance adjuster to increase RF energy to be transferred to the tissue or decrease a phase difference between the measured current and voltage. The impedance adjuster may include a variable capacitor connected in series to the RF circuit. While the variable capacitor is adjusted to reduce a loss of RF energy, after identifying change in the loss of the RF energy through the sensor while controlling capacitance of the variable capacitor to be increased or decreased.

Further, the controller performs control to carry out a first treatment mode where the RF electrode is inserted in a dermal layer and transfers RF energy, or a second treatment mode where the RF electrode is inserted in a fat layer and transfers RF energy, based on a user's settings, and the impedance adjuster may be controlled to operate in the second treatment mode.

Further, the controller may perform control to carry out an adjustment mode where RF energy is transferred to the body tissue to adjust the impedance adjuster, and a treatment mode where the adjusted impedance adjuster is used to transfer RF energy to the body tissue, and RF energy provided through the RF generator in the adjustment mode may be controlled to be smaller than RF energy provided in the treatment mode.

Meanwhile, the object of the disclosure is achieved by providing a control method based on a RF treatment device, including: inserting an RF electrode into body tissue; transferring RF energy, which is provided from an RF generator to the RF electrode along an RF circuit, to the body tissue; detecting a loss of RF energy due to impedance characteristics of the body tissue while the RF energy is being transferred to the body tissue; adjusting impedance of an impedance adjuster provided on the RF circuit to reduce the loss of the RF energy; and transferring the RF energy to the body tissue by providing the RF energy to the RF electrode through the RF circuit of which the impedance is adjusted.

Further, the object of the disclosure is achieved by providing a medical RF device including: a plurality of RF electrodes configured to be selectively inserted in body tissue and transfer RF energy generated in an RF generator to the body tissue, a sensor configured to measure RF parameters while RF energy is being transferred to the body tissue and detect information related to impedance of the body tissue based on the measured RF parameters, and an identifier configured to identify a patient's tissue characteristics based on comparison between information detected in the sensor and previously stored reference data. In this case, the plurality of RF electrodes may be inserted into a tissue layer in which collagen is distributed in skin tissue and identify the patient's tissue characteristics.

Further, the RF parameters measured by the sensor may include at least one of power supplied from the RF generator, and voltage and current applied to the body tissue through the RF electrodes. Specifically, the sensor may detect a value corresponding to a phase difference between RF voltage and RF current generated by impedance characteristics of the body tissue based on the measured RF parameters, and the identifier may identify a patient's tissue characteristics based on the value detected in the sensor. In this case, the sensor may derive a value corresponding to the phase difference, based on power supplied from the RF generator and the RF energy power calculated based on the measured voltage and current.

The identifier may be configured to identify an aging degree of the tissue based on comparison between information detected in the sensor and the reference data. Specifically, the identifier may identify the more the phase difference, the older the measured tissue.

Further, the object of the disclosure is achieved by providing a medical RF device including: an RF generator configured to generate measurement RF energy for detecting characteristics of tissue and treatment RF energy for treating the tissue, a plurality of RF electrodes configured to be selectively inserted in the body tissue and transfer the measurement RF energy and the treatment RF energy from the RF generator to the body tissue, a sensor configured to measure RF parameters while the measurement RF energy and the treatment RF energy are being transferred to the body tissue and detect information related to impedance of the body tissue based on measured RF parameters, an identifier configured to identify a patient's tissue characteristics based on comparison between the information related to the impedance detected while the measurement RF energy is being transferred and the previously stored reference data, and an impedance adjuster provided on the RF circuit and controlled to reduce a loss of energy transferred to the body tissue based on the information related to the impedance detected while the treatment RF energy is being transferred.

Further, the object of the disclosure is achieved by providing an RF based tissue examination method including: inserting an RF electrode into a patient's body tissue, transferring RF energy generated by an RF generator to the body tissue through the RF electrode, measuring RF parameters while RF energy is being transferred to the body tissue and detecting information related to impedance of the body tissue based on the measured RF parameters, and identifying a patient's tissue characteristics based on comparison between the detected information and the previously stored reference data.

### Advantageous Effects

According to the disclosure, it is possible to transfer enough energy in spite of various characteristics of the tissue because a loss of radio frequency (RF) energy transferred to tissue due to impedance characteristics of the tissue is reduced, thereby improving a treatment effect.

Further, a treatment device autonomously detects characteristics of tissue and transfers optimal RF energy, so that a user can carry out appropriate treatment without accumulated know-how even when the user lacks treatment experience.

Further, according to the disclosure, characteristics of tissue are identified based on given RF parameters to thereby quantify and continuously monitor a patient's tissue information, and it is thus possible to carry out optimal treatment as the quantified tissue information is taken into account at treatment to select a treatment parameter.

### DESCRIPTION OF DRAWINGS

FIG. 1 is a perspective view of a medical radio frequency (RF) device according to a first embodiment of the disclosure,

FIG. 2 is a perspective view of a handpiece in the medical RF device of FIG. 1,

FIG. 3 is a block diagram of a main control system in the medical RF device of FIG. 1,

FIG. 4 is a schematic circuit diagram of an RF circuit formed when tissue is treated by a conventional RF treatment device,

FIG. 5 is a graph showing power based on voltage and current having a predetermined phase difference,

FIG. 6 is a schematic circuit diagram of an RF circuit in the RF treatment device of FIG. 1,

FIG. 7 is a perspective view illustrating an impedance adjuster of FIG. 3,

FIG. 8 illustrating treatment based on a first treatment mode of the RF treatment device,

FIG. 9 illustrates treatment based on a second treatment mode of the RF treatment device,

FIG. 10 is a flowchart showing a method of controlling the RF treatment device of FIG. 1,

FIG. 11 is a detailed flowchart showing an adjusting operation of FIG. 10,

FIG. 12 is a block diagram of a main control system in an RF examination device according to the second embodiment,

FIG. 13 is a graph showing a phase difference between voltage and current according to conditions of tissue,

FIG. 14 is a flowchart showing a method of controlling the RF examination device of FIG. 12,

FIG. 15 is a front view showing an end portion of a handpiece in a medical RF device according to a third embodiment,

FIG. 16 is a flowchart showing a method of controlling a medical RF device according to a fourth embodiment, and

FIG. 17 is a perspective view of a medical RF device according to a fifth embodiment.

### MODE FOR CARRYING OUT DISCLOSURE

Below, a medical radio frequency (RF) device and a method of controlling the same according to an embodiment of the disclosure will be described in detail with reference to the accompanying drawings. In the following, a relationship between positions of elements will be fundamentally described based on the drawings. For convenience of description, the elements in the drawings may be simplified or exaggerated as necessary. Therefore, the disclosure is not limited to the following description, but may be embodied by adding, modifying or excluding various devices.

Hereinafter, the ‘medical RF device’ refers to any device using RF energy for medical purposes. The medical RF device may include an RF treatment device for tissue treatment and an RF examination device for examining characteristics of tissue, and may include various devices using RF energy for medical purposes.

Hereinafter, the ‘RF treatment device’ refers to any device for treating mammals including humans. The treatment device may include various devices for treatment, which transmit RF energy for the purpose of improving conditions of a lesion or tissue. In the following embodiments, description will be made focusing on a device for treatment of a dermal lesion. However, the disclosure is not limited to these embodiments, but may be applied to various devices used in transferring RF energy to various affected areas, such as a device for surgical treatment of an internal organ lesion.

Hereinafter, the ‘RF examination device’ refers to any device for examining characteristics of tissue (for example, health conditions, collagen content, water content, etc. of tissue) of mammals including humans. The RF examination device includes various devices that use RF pulses to examine the characteristics of the tissue, but may be applied to various devices used in examining various characteristics of internal organ tissue as well as dermal tissue like a treatment device.

Hereinafter, the ‘tissue’ refers to a set of cells that make up various body organs of animals including humans, and includes various tissues that make up various organs in a body, such as dermal tissue.

Hereinafter, the ‘insert’ refers to an element, which is inserted into tissue, in the treatment device. The insert includes various elements, an end portion of which has a sharp, thin and long structure like those of a needle, a micro-needle and a catheter and is inserted up to the inside of tissue by penetrating the surface of the tissue.

Further, an RF circuit will be illustrated and described as simplified focusing on major elements and major factors. Therefore, various circuit elements may be additionally included in the RF circuit besides the illustrated or mentioned elements. However, elements that have relatively little effect or similar effect between control groups will not be described or will be described on the assumption that they have no effects.

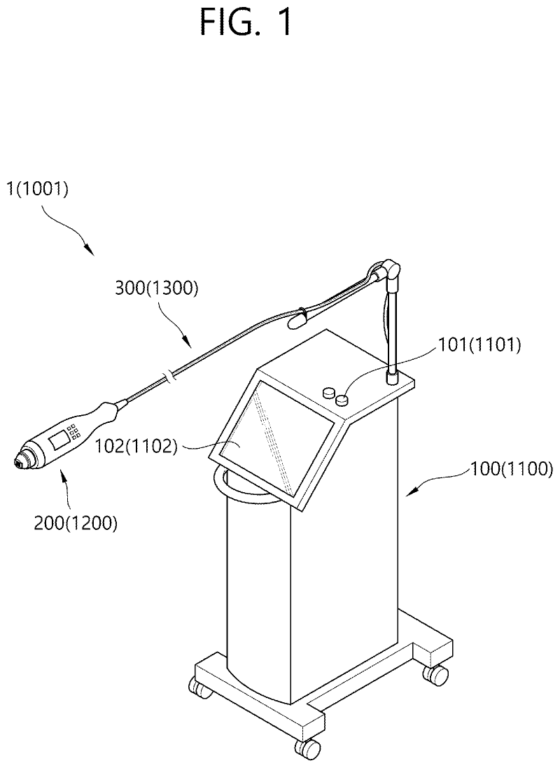

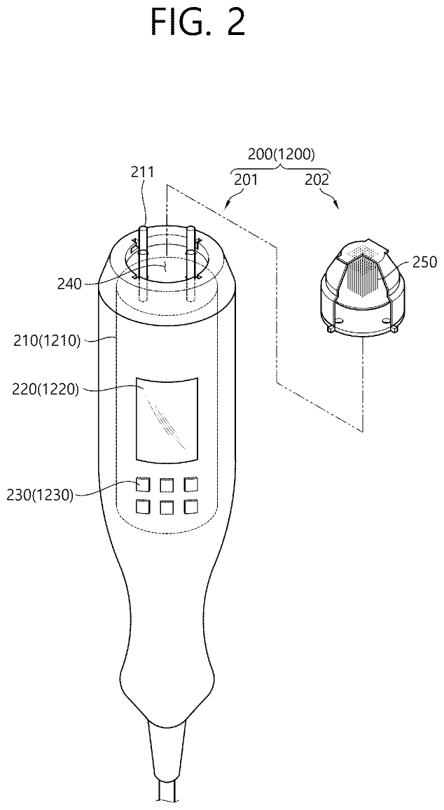

Below, a medical RF device according to a first embodiment of the disclosure will be described with reference to FIG. 1. FIG. 1 is a perspective view of the medical RF device according to the first embodiment of the disclosure, and FIG. 2 is a perspective view of a handpiece in the medical RF device of FIG. 1.

As shown in FIG. 1, the medical RF device according to this embodiment includes an RF treatment device. Such an RF treatment device 1 includes a main body 100, and a handpiece 200 to carry out treatment while being gripped by a user.

The main body 100 internally includes an RF generator 110. The RF generator 110 generates RF energy used for treatment. The RF generator 110 may generate the RF energy having various parameters (for example, power, pulse duration, pulse interval, frequency, etc.) according to a patient's habitus, treatment purposes, treatment parts, etc. The RF energy generated in the RF generator according to this embodiment is generally used for the purpose of tissue treatment. However, the RF energy may be used for detecting the characteristics of tissue or a circuit besides the purpose of tissue treatment. In this regard, detailed descriptions will be made later.

The main body 100 externally includes various switches 101 and a display 102. The switch 101 is configured to control operations of the treatment device as well as power on/off, and the display 102 includes a display device to display various pieces of information such as information about operations of the treatment device. The display 102 may be embodied by a touch screen configured to not only display various pieces of information but also allow a user to set treatment details in person through the display 102.

The handpiece 200 is connected to the main body 100 by a connector 300. The connector 300 is configured to transmit power, a control signal, etc. required for operating various devices of the handpiece 200 from the main body 100. For example, the RF energy generated in the RF generator 110 of the main body 100 is transferred to the handpiece 200 through the connector 300. The connector 300 may be embodied by a cable including various signal lines, power lines, etc. or may be configured to have a curved structure to be easily curved by a user's control.

Meanwhile, the handpiece 200 is configured to carry out treatment as disposed at a treatment position, and shaped to be used while being gripped by a user's hand. The handpiece 200 includes an insert 250 to be selectively inserted into tissue to carry out invasive treatment, a driver 210 for moving the insert 250, and a handpiece controller 230 for controlling operation details of the insert 250 and the driver 210.

Specifically, as shown in FIG. 2, the handpiece controller 230 and a handpiece display 220 are provided on an outer surface of a housing that forms a body 201 of the handpiece 200. The handpiece controller 230 is configured to turn on/off the handpiece 200, adjust an insertion depth of the insert 250, and adjust a level or the like of energy to be transferred through the insert 250. The handpiece display 220 displays various pieces of information needed for treatment to a user. Therefore, a user controls the handpiece controller 230 while gripping the handpiece 200 in his/her hand, thereby carrying out the treatment and at the same time checking the treatment details through the display 220.

The handpiece 200 internally includes the driver 210. The driver 210 is configured to move the insert 250 so that the insert 250 can be selectively inserted into tissue and withdrawn from the tissue. The driver 210 may be embodied using various linear actuators such as a solenoid, hydraulic/pneumatic cylinders, etc., a linear motor, etc. As an example, the driver 210 in this embodiment drives output terminals 211 provided at one side to linearly move in a lengthwise direction. A plurality of needles corresponding to the insert 250 is provided at an end portion of the output terminals 211, and the insert 250 may appear and disappear at one end (one end to be in contact with the treatment position) of the handpiece 200 as the output terminals 211 linearly move.

The insert 250 is, as described above, configured to be inserted up to the inside of the tissue by penetrating the surface of the tissue. The insert 250 in this embodiment is achieved as a microneedle that can be easily inserted in the tissue, but may be embodied to have various structures such as a single needle structure, a catheter, etc. besides the microneedle. The microneedle in this embodiment may include a needle having a diameter ranging from several to thousands of/GM, preferably, a diameter of 10 to 1000 μm.

The end portion of the insert 250 including a plurality of microneedles is formed with RF electrodes 251. The RF electrodes 251 are connected to the RF generator 110 by the foregoing RF circuit, the RF energy generated in the RF generator is provided to the RF electrodes 251 along the RF circuit. Therefore, the RF electrodes 251 transfer the RF energy to the inside of the tissue while being inserted in the tissue. In this case, the insert 250 is formed with a conductive path along a lengthwise direction, and covered with an insulating material on an outer surface except the end portion. Therefore, the RF energy is transferred through only the end portion of the insert 250 in which the RF electrodes 251 are formed.

In this embodiment, the insert is embodied by a tip module 202 detachably mounted to a handpiece end portion, and replaceable after treatment. The tip module 202 includes the plurality of microneedles, and detachably mounted to a recessed portion 240 at one end of the handpiece body. The output terminals 211 are positioned on the rear of the tip module 202, and the plurality of microneedles accommodated in the tip module moves forward/backward as the output terminals 211 moves forward/backward. Further, when the tip module is mounted to the recessed portion 240, the microneedles of the tip module are electrically connected to the RF circuit in the handpiece so that the RF energy can be transferred to the inside of the tissue through the RF electrodes 251 of the microneedles.

Detailed structures of the handpiece and the tip module may be variously embodied with reference to those disclosed in Korean patent publication No. 10-1300123 or the like disclosure.

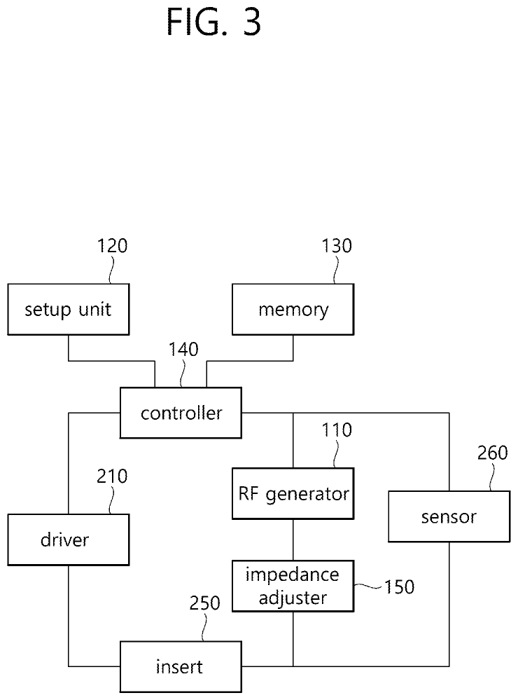

FIG. 3 is a block diagram of a main control system in the medical RF device of FIG. 1. Below, a control structure of a medical RF device according to an embodiment will be described in detail with reference to FIG. 3.

A controller 140 is configured to control operations of various elements of the main body 100 and the handpiece 200. As shown in FIG. 3, the controller 140 controls the operations of the driver 210, thereby inserting the insert 250 into the tissue, withdrawing the insert 250 from the tissue, and controlling an insertion depth of the insert 250. Further, the controller 140 may control the RF generator 110 to adjust on/off of RF pulses and the parameters of the RF pulses. Thus, the RF treatment device 1 can provide RF pulses having appropriate parameters after inserting the microneedles into the tissue.

A setup unit 120 is configured to allow a user to set up a treatment mode and treatment details. The controller 140 controls various elements to carry out treatment based on settings set up through the setup unit 120. The setup unit 120 may be embodied by the foregoing display and/or switch. Therefore, when various setting options are displayed through the display 102, a user selects the option by touching the display or controlling the switch to thereby change the settings.

Further, the RF treatment device 1 additionally includes a memory 130 in which various pieces of data are stored. The controller 140 may store information needed for controlling the RF treatment device in the memory 130, or load the stored data from the memory 130 and use the data in control.

Furthermore, the RF treatment device 1 additionally includes a sensor 260 and an impedance adjuster 150. Here, the sensor 260 measures various parameters of RF energy transferred through the RF circuit while the RF energy is transferred to body tissue. Therefore, various pieces of information such as the characteristics of the RF energy transferred to the body tissue, the impedance characteristics of the body tissue, etc. may be obtained. The impedance adjuster 150 is provided on the RF circuit, and includes circuit elements for adjusting impedance. The controller 140 controls the impedance adjuster 150 based on information measured by the sensor 260.

Below, the foregoing configurations of the sensor 260 and the impedance adjuster 150 will be described in more detail with reference to the drawings.

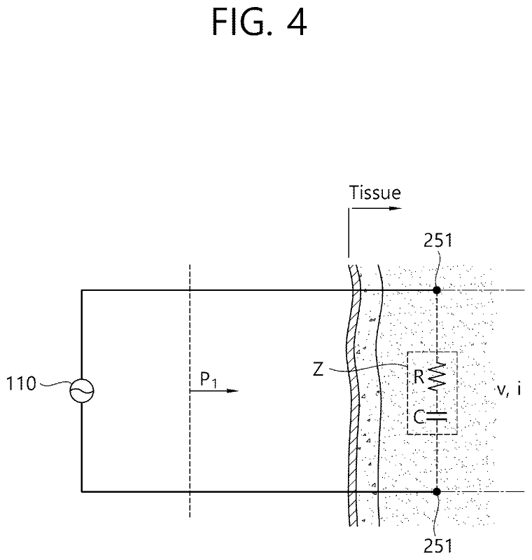

FIG. 4 is a schematic circuit diagram of a conventional RF treatment device and an RF circuit formed in tissue when the tissue is treated by the conventional RF treatment device. As shown in FIG. 4, the RF energy generated by the RF generator 110 is provided to an RF electrode along an RF circuit, and thus the RF energy is transferred to body tissue. In this case, the body tissue has resistance R, and therefore the RF energy is converted into thermal energy while passing through the tissue, thereby transferring the thermal energy to the body tissue.

However, the impedance of the body tissue may include capacitance C in addition to the resistance R. The capacitance C causes repetitive power exchange between electric fields formed inside the tissue by the RF generator and the capacitance, thereby hindering the RF energy from being converted into thermal energy and the like nonelectric energy. Therefore, the amount of RF energy transferred to the body tissue may be varied depending on the impedance characteristics of the tissue even though the RF generator provides the same RF energy.

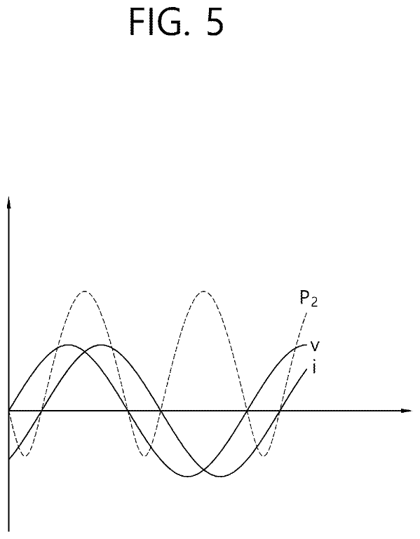

FIG. 5 is a graph showing power based on voltage and current having a predetermined phase difference. Specifically, the capacitance C among the impedance characteristics of the body tissue causes a phase difference between voltage v and current i. In this case, power P actually transferred to tissue is based on the product of voltage and current. When there is a phase difference between voltage v and current i as shown in FIG. 5, the power P transferred to the body tissue is decreased as compared with a case where voltage and current are in phase with each other.

Here, the impedance characteristics of the body tissue Z is varied depending on components of tissue. Fatty tissue has higher capacitance C than the other tissue. It is expected that the high capacitance C is because the fat layer contains less water and has dielectric characteristics. Therefore, when RF treatment is targeted for the fat layer, a loss of energy transferred to the body tissue is greater than that of when the other tissue is treated. In result, the treatment is highly likely to be carried out with intensity that is not as high as needed for treatment of the fat layer.

Therefore, the RF treatment device 1 according to an embodiment is configured to detect the transfer characteristics of RF energy through the sensor 260 while the RF energy is transferred, and reduce the foregoing loss of the RF energy through the impedance adjuster 150.

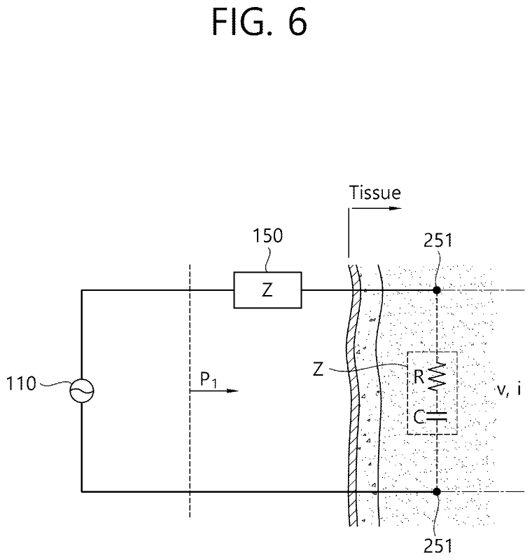

FIG. 6 is a schematic circuit diagram of an RF circuit in the RF treatment device of FIG. 1. As shown in FIG. 6, the RF energy generated in the RF generator 110 is transferred to the RF electrodes 251 through the RF circuit. FIG. 6 illustrates two RF electrodes as an equivalent circuit for convenience of description, but a plurality of RF electrodes are actually used in transferring the RF energy. Further, FIG. 6 illustrates that the RF generator 110 and the RF electrodes 251 are connected in series, but not limited thereto. Alternatively, the RF generator and the RF electrode may be connected by at least one secondary coil structure.

The sensor 260 includes a plurality of sensors connected to the RF circuit, and detects various parameters of RF energy transferred through the RF circuit. For example, the sensor 260 detects at least one of parameters, such as power P₁ supplied from the RF generator 110, voltage v applied to the tissue through the RF electrode, and current i flowing between the RF electrodes via the tissue. Here, the power P₁ supplied from the RF generator may be power measured between both terminals of the RF generator, or power supplied to the RF electrode through a secondary coil in the case of the secondary coil structure. The sensor 260 may identify the impedance characteristics of the body tissue based on the measured parameters, and detect a loss of the RF energy transferred to the body tissue.

Specifically, the sensor 260 measures the power P₁ supplied from the RF generator 110 at a predetermined sampling period in real time. Further, RF energy E₁ transferred from the RF generator 110 to the RF electrodes 251 is periodically measured by integrating the measured power. At the same time, the sensor 260 measures voltage v and current i applied to the body tissue through the RF electrodes 251 at a predetermined sampling period in real time, and calculates power P₂ transferred to the tissue by obtaining the product of the measured voltage and current. Then, the calculated power P₂ is integrated to periodically calculate RF energy E₂ transferred to the body tissue. Here, P₁ and E₁ are power and energy desired to be actually transferred to the tissue through the RF circuit, and P₂ and E₂ are power and energy actually transferred to (absorbed in) the tissue according to the impedance characteristics of the tissue.

With such processes, the sensor 260 identifies the impedance characteristics of the tissue based on the RF energy (obtained by the measured power) E₁ supplied from the RF generator and the RF energy (obtained by the calculated power) E₂ actually transferred to the tissue. Specifically, a power factor is obtained based on a ratio of the RF energy supplied from the RF generator and the RF energy transferred to the tissue, and the capacitance C in the impedance of the tissue and a phase difference between voltage and current due to the capacitance C are identified based on the power factor. Further, the RF energy E₂ actually transferred to the tissue is smaller than the RF energy E₁ supplied from the RF generator, and such difference is identified as an energy loss caused by the capacitance component of the tissue.

Like this, when the impedance characteristics of the tissue and the energy loss due to the impedance characteristics are detected through the sensor 260, the controller 140 controls the impedance adjuster 150 in consideration of a detection result of the sensor 260. In this case, a loss of RF energy is caused by the phase difference between the voltage and the current based on the capacitance C of the tissue, and thus the controller 140 controls the impedance adjuster 150 to decrease the phase difference. Alternatively, the impedance adjuster 150 may be controlled to reduce the loss of the RF energy, i.e., increase the energy E₂ transferred to the tissue.

The impedance adjuster 150 includes at least one circuit element by which impedance is variable. For example, the impedance adjuster 150 includes a variable capacitor connected in series to the RF circuit. In this case, the impedance adjuster 150 can adjust the impedance of the RF circuit for transferring the RF energy, and thus compensate for the phase difference or energy loss due to the impedance characteristics of the tissue.

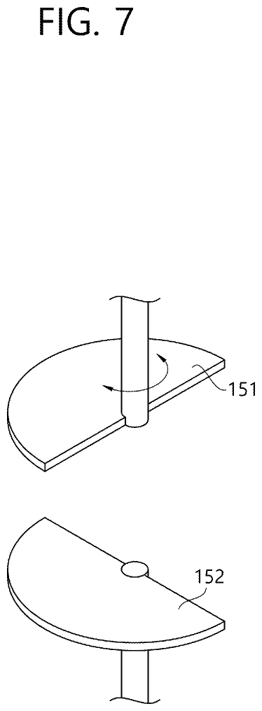

FIG. 7 is a perspective view illustrating the impedance adjuster of FIG. 3. As shown in FIG. 7, the impedance adjuster 150 includes two opposite pole plates 151 and 152, and ono pole plate 151 is rotatable. Therefore, it is possible to linearly change the capacitance by rotating one pole plate 151 to adjust an area overlapping with the other pole plate 152.

The controller 140 rotates one pole plate 151 in a direction for increasing and decreasing the capacitance of the impedance adjuster, and the sensor 260 detects change in parameters of the RF energy due to variance in the capacitance during the rotation. The controller 140 identifies an adjusting direction and an adjusting degree of the impedance adjuster to decrease the phase difference and reduce the energy loss based on the detection result of the sensor 260, and adjusts the impedance of the impedance adjuster 150 based on the identified adjusting direction and degree.

FIG. 7 shows that the impedance adjuster employs the variable capacitor, but various circuit elements may be used for the impedance adjuster. For example, there may be used various capacitors of which capacitance is adjusted by changing a gap between pole plates, or using a switch. Further, the impedance adjuster may be configured with combination of various elements such as a variable resistor.

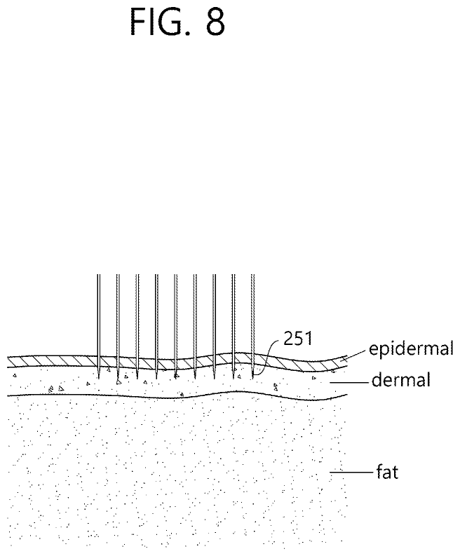

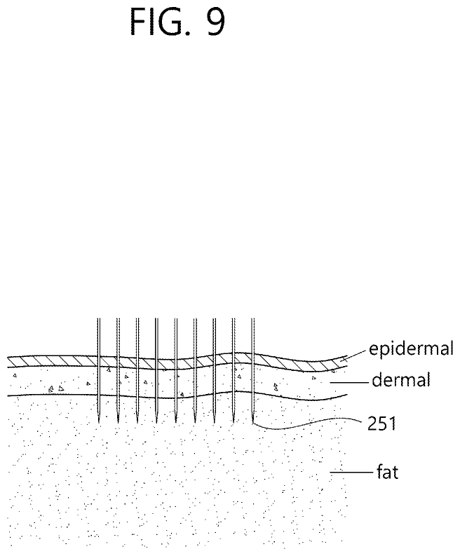

FIG. 8 illustrating treatment based on a first treatment mode of the RF treatment device of FIG. 1, and FIG. 9 illustrates treatment based on a second treatment mode of the RF treatment device of FIG. 1. In this embodiment, the RF treatment device 1 includes at least two treatment modes. Here, the first treatment mode refers to a mode for scar treatment, acne treatment, skin elasticity improvement, etc., in which the RF electrodes 251 are inserted to be positioned in a dermal layer as shown in FIG. 8 and transfer the RF energy. Further, the second treatment mode refers to a mode for fat removal, microcontouring-plasty, skin elasticity improvement, etc., in which the RF electrodes 251 are inserted to be positioned in a fat layer under the dermal layer as shown in FIG. 8 and transfer the RF energy to the fat layer.

When a user selects the treatment mode through the setup unit 120, the controller 140 controls various elements based on the selected treatment mode. For example, when the first treatment mode is selected, the controller 140 controls the driver 210 to insert the RF electrode up to a first depth corresponding to the depth of the dermal layer. Further, the RF generator 110 is controlled to provide the RF energy having a first parameter suitable for the dermal layer treatment. Further, when the second treatment mode is selected, the controller 140 controls the driver 210 to insert the RF electrode up to a second depth corresponding to the depth of the fat layer. Further, the RF generator 110 is controlled to provide the RF energy having a second parameter suitable for the fat layer treatment. Further, the controller 140 may perform control corresponding to RF parameter information detected in the sensor 250 during the treatment.

Specifically, when the first treatment mode is set up, the controller 140 may adjust the parameter of the RF energy provided by the RF generator 110 based on information detected by the sensor 260 during the treatment (first control). It is possible for the sensor 260 to in real time measure voltage and current applied to the tissue, and therefore possible to monitor resistance of the tissue and condition changes due to variance in the resistance. Because the resistance is varied depending on the positions and characteristics of the tissue and thus time taken in making the tissue reach a target condition is varied, the controller 140 may control the RF generator 110 to control the output power, pulse width, etc. of the RF energy.

Further, when the second treatment mode is set up, the controller 140 may control the impedance adjuster 150 to reduce a loss of energy transferred to the tissue based on information detected by the sensor 260 during the treatment. While the impedance of the dermal layer has a relatively small capacitance component to thereby have little effect on the energy loss, the impedance of the fat layer has a relatively large capacitance component to thereby cause a high loss of energy transferred to the tissue during the treatment. Therefore, the controller 140 in the second treatment mode selectively takes the impedance characteristics of the tissue into account to thereby control the impedance adjuster 150. Therefore, it is possible to reduce the energy loss that occurs in the second treatment mode (second control).

The first control takes a resistance component among the impedance characteristics of the tissue into account to control the output transferred from the RF generator, and is thus different from the second control that takes a capacitance component among the impedance characteristics of the tissue into account to control capacitance of variable impedance. The foregoing description shows that the first control is performed in the first treatment mode, and the second control is performed in the second treatment mode, but, as necessary, both the first control and the second control may be performed in the first treatment mode and both the first control and the second control may be performed in the second treatment mode.

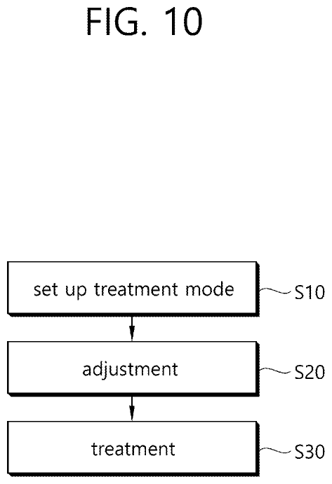

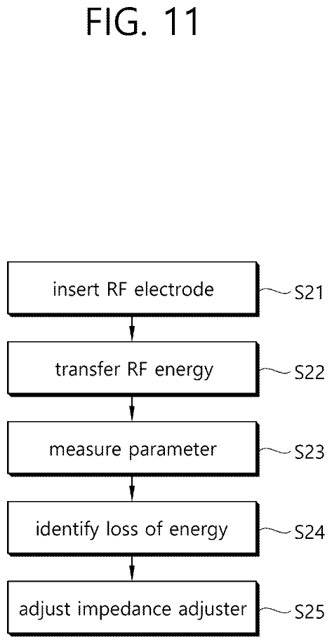

FIG. 10 is a flowchart showing a method of controlling the RF treatment device of FIG. 1, and FIG. 11 is a detailed flowchart showing an adjusting operation of FIG. 10. Below, the method of controlling the treatment device according to an embodiment will be described with reference to FIGS. 9 and 10.

Before carrying out the treatment, a user sets up treatment details through the setup unit 120 (S10). In this operation, a user sets up the treatment mode and various parameters in consideration of treatment position, a treatment lesion and a patient's condition. In this case, a user may select one of the first treatment mode and the second treatment mode, and the following description will be made focusing on the second treatment mode by way of example.

First, as described above, characteristics of tissue in the fat layer are taken into account to perform an operation of adjusting the impedance of the RF circuit (S20). First, a user puts the handpiece 200 at a first position. Here, the first position may be a separate examination position for performing the adjusting operation, or may be the first treatment position. The controller 140 operates the driver in response to a user's control, and inserts the plurality of RF electrodes 251 into a fat layer of a patient (S21). Further, the RF generator 110 is controlled to transfer the RF energy to the fat layer in the body through the RF electrode (S22). In this operation, the RF energy transferred to the tissue may be provided to have a lower output or a shorter pulse width than that of the RF energy transferred in a treatment operation to be described later, so as to prevent desiccation of tissue. When the RF energy higher than a predetermined level is applied to the tissue, the impedance is suddenly varied with a desiccation progress of the tissue, and it is therefore difficult to use the impedance in identifying the characteristics of the tissue

While the RF energy is being transferred in this operation, the sensor 260 measures various parameters of the RF energy transferred along the RF circuit (S23). For example, the sensor 260 may measure power P₁ supplied from the RF generator 110, and voltage v and the current i transferred through the RF electrodes 251. Further, as described above, the measured power P₁ is compared with the power P₂ calculated based on the measured voltage and current, thereby detecting the impedance characteristics of the tissue and a loss of energy (S24). With this, the controller 140 controls the variable capacitor of the impedance adjuster 150 (S25). In this case, the sensor 260 continuously detects the energy loss in the RF circuit due to the capacitance variance of the variable capacitor. Specifically, the controller 140 detects the loss of the RF energy through the sensor while increasing the capacitance of the variable capacitor (first step), and then detect the loss of the RF energy while, reversely, decreasing the capacitance (second step). Further, the controller adjusts the variable capacitor to reduce the loss of the RF energy based on results detected through the first and second steps. With these steps, the RF circuit is controlled to reduce an energy transfer loss and a phase difference between the voltage and the current caused by the tissue characteristics of the fat layer during the treatment.

When the impedance control is applied to the RF circuit, treatment operation is performed (S30). A user changes the position of the handpiece 200 into a second position. The controller 140 operates the driver 210 in response to a user's control, and thus inserts the RF electrode up to the fat layer, thereby transferring the RF energy for the treatment to the tissue through the RF electrode. Because the RF circuit is adjusted to compensate for the capacitance characteristics of the tissue through the adjusting operation, the treatment is carried out in this treatment operation while transferring energy as much as desired to the tissue.

Further, the sensor 260 in the treatment operation may detect the impedance (resistance) of the tissue during the treatment, and then control the power of the RF energy generated by the RF generator 110 based on the detected resistance (see the foregoing first control).

According to an alternative embodiment, the foregoing first control may be performed in the treatment operation. In other words, the sensor may measure the resistance of the tissue during the treatment, and the power of the RF generator may be controlled by reflecting the measured resistance. Such power control of the RF generator may be immediately reflected to the second position based on a result sensed at the second position, or may be reflected at third position treatment based on the result sensed at the second position.

When the second position treatment is completed by the foregoing operations, a user changes the position of the handpiece into third and fourth treatment positions and performs the treatment operation at each changed position.

The foregoing embodiment shows that the first position is an examination position where the adjusting operation is performed, and the treatment starts from the second position, but the disclosure is not limited to this embodiment. When the RF energy is sufficiently transferred to the first position by the adjusting operation, the treatment may start from the first position. Further, the adjusting operation is performed at the first operation, and then the RF energy for the treatment is transferred through the adjusted RF circuit to thereby perform the treatment at the first position.

Further, the foregoing embodiment shows that the first control is performed in the treatment operation, but the disclosure is not limited to this embodiment. Alternatively, both the first control and the second control may be performed in the adjusting operation.

Below, a medical RF device according to a second embodiment of the disclosure will be described with reference to the drawings. The medical RF device according to the second embodiment refers to a biopsy device using an RF (hereinafter, referred to as an RF examination device). According to this embodiment, the RF examination device 1001 refers to a device for using the RF energy to measure the condition or characteristics of the tissue, which is distinguished from the RF treatment device according to the foregoing embodiment. However, the RF examination device includes elements similar to those of the RF treatment device according to the foregoing embodiment because it transfers the RF energy to the tissue, and measures the conditions or characteristics of the tissue based on RF parameters measured while the RF energy is transferred.

Accordingly, the RF examination device 1001 according to this embodiment will be described on the assumption that the same names will be given to elements similar to those of the foregoing embodiments, and the same technical features of the elements will be replaced by the description of the foregoing embodiments to avoid repetitive descriptions. However, the RF examination device set forth herein is merely an embodiment and is variously modifiable without being limited to the following embodiment.

The RF examination device 1001 according to this embodiment includes a main body 1100, a handpiece 1200, and a connector 1300 for connecting the main body and the handpiece (see FIGS. 1 and 2). Further, the main body internally includes an RF generator 1110 to generate an RF pulse, and externally includes a switch 1101 and a display 1102 on an outer surface thereof to control operations of the examination device or to display various pieces of information to a user.

Further, the handpiece 1200 measures the characteristics of the tissue at a position adjacent to the tissue to be examined. The handpiece 1200 in this embodiment includes an insert 1250, a driver 1210 and an operator 1220 for controlling the insert 1250 and the driver 1210 like those of the foregoing embodiment. The insert 1250 has a tip module structure including microneedles as shown in FIGS. 1 and 2, and is connected to the RF generator, thereby transferring the RF energy for measurement used in examining the characteristics of the tissue. Further, the driver 1210 drives the insert 1250 to move backward so that the insert 1250 can be retractable into the tissue to examine the tissue.

However, the mechanical structure, the driving method, the RF energy transferring system, etc. of the main body and the handpiece are similar to those of the foregoing RF treatment device according to the first embodiment, and therefore repetitive descriptions thereof will be avoided.

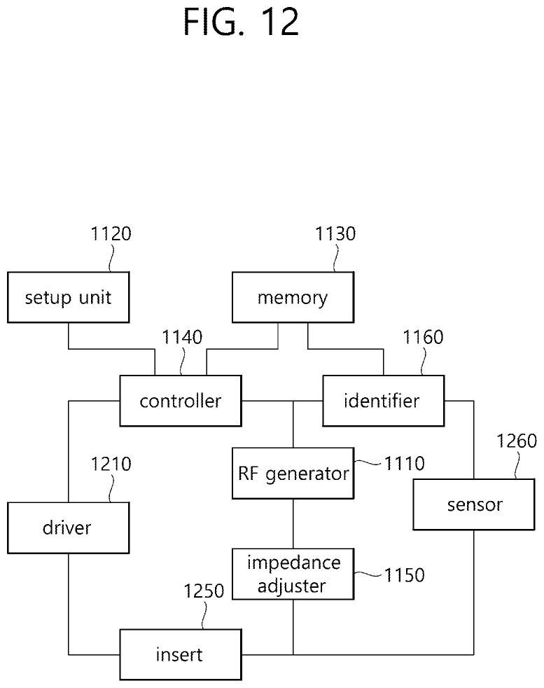

FIG. 12 is a block diagram of a main control system in an RF examination device according to the second embodiment of the disclosure. Below, a control structure of the RF examination device according to this embodiment will be described in detail with reference to FIG. 12.

The controller 1140 is configured to control operations of various elements in the main body and the handpiece like those of the foregoing embodiment. Therefore, the controller 1140 controls the driver 1210 to insert the insert 1250 into the tissue, and controls the RF generator 1110 to generate the RF energy needed for examination.

A setup unit 1120 is configured to allow a user to set up examination details. A user may set up an examination pattern, examination times, etc. through the setup unit 1120, and the controller 1140 controls various elements to perform an examination operation based on the setup details. A memory 1130 is configured to store various pieces of data to be used in examination. Therefore, the controller 1140 may store needed information in the memory 1130, or control various elements based on the data stored in the memory 1130.

A sensor 1260 is configured to measure various parameters of the RF energy transferred to the tissue during the examination. The sensor 1260 may be configured to detect information related to impedance of body tissue based on the measured parameters.

An identifier 1160 is configured to identify a patient's tissue characteristics based on information detected by the sensor 1260. FIG. 12 illustrates that the identifier 1160 is a separate element branched into the controller 1140 or the sensor 1260, but the identifier may be provided as a subordinate element of the controller or as a subordinate element of the sensor.

As described above in the foregoing embodiment with reference to FIGS. 4 and 5, an impedance component of tissue may include both a resistance component and a capacitance component, and a phase difference between current and voltage applied to the tissue occurs according to magnitudes of capacitance components.

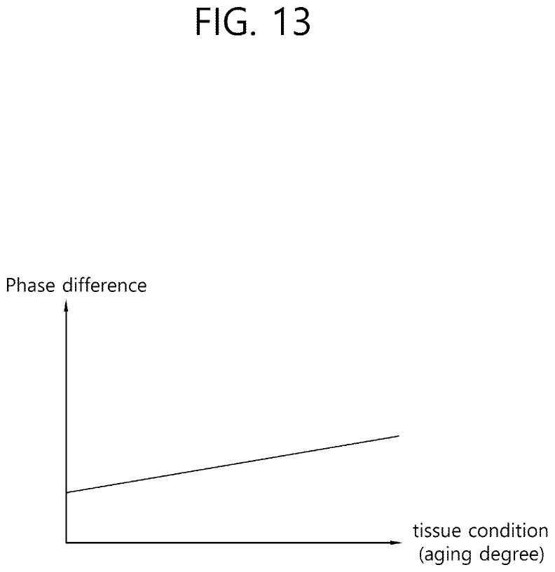

FIG. 13 is a graph showing a phase difference between voltage and current according to conditions of tissue. As a result of experiments with a plurality of patient groups on skin tissue, the phase difference between voltage and current applied to the tissue was varied depending on patients even though RF energy of the same power was applied to equivalent tissue (for example, a dermal layer). In particular, the younger the patient, the lower the phase difference between the voltage and the current applied to the tissue. In a similar age group, the better the skin tissue condition, the lower the phase difference. Specifically, a relationship between a good condition of skin tissue (for example, an aging degree) and the phase difference between the voltage and the current applied to the tissue is as shown in FIG. 13. It is identified that such a relationship is based on water content in tissue and fat cell distribution of collagen tissue. In other words, the tissue with higher water content and fewer fat cells makes it more difficult to allow the tissue function as a capacitor, thereby decreasing a capacitance component and thus lessening a phase difference between the applied voltage and current. On the other hand, when it is taken into account that a representative characteristic of aged tissue is dehydration, the more the skin is aged, the greater the phase difference is exhibited under the same condition the more the skin is aged. Therefore, the RF examination device according to this embodiment can identify the characteristics of the tissue based on the parameter measured by transferring RF energy for measurement.

Specifically, the controller 1140 drives the driver 1210 to insert the insert 1250 into tissue to be examined, and drives the RF generator 1110 to transfer the RF energy for the measurement to the inside of the tissue through the RF electrodes, thereby carrying out the examination. In this case, the RF energy for the measurement to be transferred for the examination may be provided to have a lower output or a shorter pulse width than that of the RF energy transferred for treatment, so as to prevent the desiccation of the tissue. When the RF energy higher than a predetermined level is applied to the tissue, the impedance is suddenly varied with a desiccation progress of the tissue, and it is therefore difficult to use the impedance in identifying the characteristics of the tissue

Meanwhile, the sensor 1260 measures the RF parameter and obtains an indication value while the RF energy for the measurement is being transferred to the tissue. Here, the indication value refers to various values related to the phase difference and thus capable of indicating the characteristics of the tissue as a value. Such an indication value may be calculated using the measured RF parameter.

For example, like the foregoing embodiment, the sensor 1260 measures power P₁ supplied from the RF generator, and voltage v and current i applied to the tissue through the RF electrodes. Further, a value corresponding to a power factor may be obtained based on power P₂ obtained by calculating the measured voltage and current, and measured power P₁. The power factor is a cosine value of a phase difference 8 between the voltage and the current, which may be employed as the indication value. However, besides, various values drawn using the RF parameter and having a functional relation with the phase difference may be used as the indication value.

The identifier 1140 identifies the characteristics of the tissue based on the power factor obtained by the sensor 1260. The memory 1130 in this embodiment is configured to store reference data that includes information about skin tissue conditions corresponding to the indication values. Therefore, the identifier 1140 compares the obtained power factor and the reference data stored in the memory 1130, and classifies the characteristics of the tissue. The classification may be achieved by various methods of scoring health conditions of tissue, calculating a tissue age based on comparison with average phase-difference data according to ages, etc. Further, an identification result of the identifier 1140 is displayed on the display 1102, so that a user can check the condition of the tissue through the display 1102.

The foregoing tissue characteristic examination may be achieved by identifying the characteristics of the tissue based on a single measurement result from one patient, or by identifying the characteristics of the tissue based on results from examination performed at a plurality of positions.

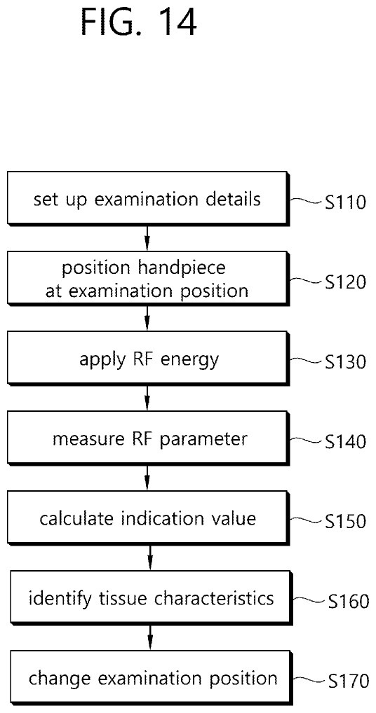

FIG. 14 is a flowchart showing a method of controlling the RF examination device of FIG. 12. Below, the method of controlling the examination device according to an embodiment will be described in detail with reference to FIG. 14

Before performing the examination, a user sets up examination details through the setup unit 1120 (S110). In this operation, a user sets up examination times, power of the RF energy for measurement, or etc. in consideration of a patient's characteristics.

When the setup operation is completed, a user puts the handpiece 1200 on a surface of tissue to be examined (S120). In an examination method according to this embodiment, the examination is performed by inserting the inserts at a plurality of positions. In this operation, the handpiece is positioned at an initial examination position, i.e., a first examination position.

When the handpiece 1200 is positioned at the first examination position, the controller 1140 inserts the insert into the tissue in response to a user's control, and transfers first measurement RF energy to body tissue through the RF electrode 1251 (S130). The insert may be controlled to be withdrawn from the tissue after transferring the first measurement RF energy.

Meanwhile, the sensor 1260 measures parameters of the RF circuit while the first measurement RF energy is being transferred (S140). Further, the sensor 1260 calculates the indication value, which corresponds to the phase difference between the voltage and the current applied to the tissue, based on the measured parameter (S150). Here, the indication value may be various values having a functional relation with the phase difference, for example, a power factor of the RF energy absorbed into the tissue. Specifically, the power P₁ supplied from the RF generator, and the voltage v and the current i measured by the sensor are measured. Further, the power factor may be calculated based on a ratio of the measured power P₁ and the power P₂ calculated from the measured voltage v and current i.

When the indication value is obtained through the foregoing operation, the identifier 1140 identifies the characteristics of the tissue by comparing the indication value with the reference data stored in the memory 1130 (S160). In this case, the lower the phase difference, in other words, the higher the power factor corresponding to the indication value, the better the tissue condition. Further, the identification results of tissue conditions may be displayed on the display 1102 for a user.

When the characteristics of the tissue are completely examined at a first position, the examination position is changed into a second position and the examination is performed at the second position (S170). Further, the foregoing operations S130 to S160 are repeated. FIG. 14 shows that the characteristics of the tissue are identified at each individual position. Alternatively, the identification values may be obtained at a plurality of positions when tissue characteristic examination is performed at the respective positions, and the identification values obtained at all examination positions may be synthetically taken into account to thereby identify and display a patient's tissue characteristics.

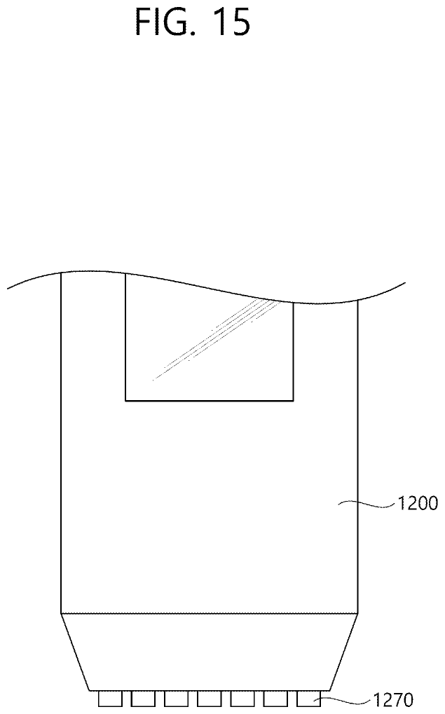

FIG. 15 is a front view showing an end portion of a handpiece in a medical RF device according to a third embodiment of the disclosure. Below, the medical RF device according to the third embodiment of the disclosure will be described with reference to FIG. 15.

In this embodiment, the medical RF device is embodied as the RF examination device. However, the foregoing RF examination device according to the second embodiment is configured to apply the measurement RF energy while the RF electrodes are inserted in the tissue, but the RF examination device according to this embodiment is configured to examine the characteristics of the tissue by applying the measurement RF energy while the RF electrodes are in contact with the surface of the tissue to be examined. When it is taken into account that a capacitance component is generally affected by the inner condition of the tissue rather than the surface of the tissue, the invasive examination method according to the second embodiment can get a more accurate result. However, when convenience of examination and a patient's pain are taken into account, the RF examination device may be configured to perform a contact examination method according to this embodiment.

In this case, as compared with the RF examination device according to the second embodiment, the handpiece does not separately include the insert and the driver, but instead includes an electrode portion 1270 provided at the end portion of the handpiece 1200 and to be in contact with the surface of the tissue. Further, the electrode portion 1270 is used to apply the measurement RF energy to the surface of the tissue, and measure the RF parameter at this time, thereby examining the characteristics of the tissue.

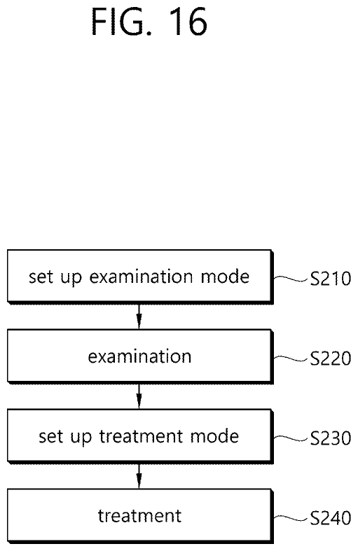

Below, a medical RF device according to a fourth embodiment of the disclosure will be described with reference to FIG. 16.

The foregoing first and second embodiments show that the RF treatment device and the RF examination device are respectively provided as separate devices. However, the RF treatment device according to the first embodiment and the RF examination device according to the second embodiment include similar elements, and therefore the medical RF device according to this embodiment is configured to perform both the examination and the treatment as the RF treatment device according to the first embodiment and the RF examination device according to the second embodiment are configured as a single device.

Specifically, the RF treatment device according to this embodiment (which can perform both the examination and the treatment, but is called the RF treatment device because it may be used to perform the examination as a previous step of the treatment) may be configured to have a structure corresponding to the RF examination device according to the second embodiment (see FIGS. 1, 2 and 12). Further, a user is allowed to select an examination mode or the treatment mode through the setup unit 1120. Therefore, when a user selects the treatment mode through the setup unit 1120, major elements such as the setup unit 1120, the memory 1130, the controller 1140, the RF generator 1110, the insert 1250, the driver 1210, the sensor 1260, etc. may be configured to operate as described in the first embodiment. Further, when a user selects the examination mode through the setup unit 1120, the major elements may be configured to operate as described in the second embodiment. However, the structures and operations of these elements have been described in detail in the first and second embodiments, and thus detailed descriptions thereof will be replaced by the descriptions of the first and second embodiments to avoid repetitive descriptions.

FIG. 16 is a flowchart showing a method of controlling a medical RF device according to a fourth embodiment. According to this embodiment, one device may be used in not only examining the characteristics of the tissue but also carrying out lesion treatment of tissue. Preferably, as shown in FIG. 16, the characteristics of the tissue may be examined as a previous step of the treatment, and then a lesion of tissue may be treated based on the examination.

In this case, a user first selects the examination mode through the setup unit (S210). Further, the RF treatment device according to this embodiment is used to examine the characteristics of the tissue (S220). In this case, the operation of examining the characteristics of the tissue is performed by transferring measurement RF energy to the tissue, specifically, through operations S110 to S170 of FIG. 14. When the examination operation is completed, a user selects the treatment mode through the setup unit (S230). Further, a lesion of tissue is treated by the RF treatment device (S240). In this case, the operation of treating the lesion of the tissue is performed by transferring the RF energy for treatment, and an adjusting operation may be selectively involved and performed according to the treatment modes (see the first embodiment). The treatment operation may be performed, specifically, through the operations of FIGS. 10 and 11. However, the descriptions about the operations shown in FIGS. 11 and 14 are replaced by the foregoing descriptions about the first and second embodiments.

With this, the characteristics of the tissue are examined as a previous step of the tissue treatment, so that a result of measuring the characteristics of the tissue can be reflected in the treatment of the tissue.

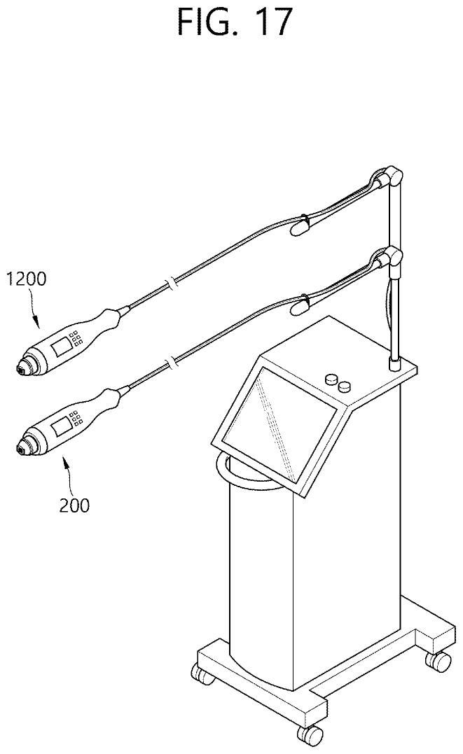

FIG. 17 is a perspective view of a medical RF device according to a fifth embodiment of the disclosure. Below, the medical RF device according to the fifth embodiment of the disclosure will be described with reference to FIG. 17.

The foregoing fourth embodiment shows that the RF examination device and the RF treatment device are provided as a single body configured to perform both the examination of the tissue and the treatment of the tissue through a single handpiece. On the other hand, the medical RF device according to this embodiment may be configured to separately include an examination handpiece 1200 used in the examination operation of the tissue and a treatment handpiece 200 used in the treatment operation of the tissue, while the RF examination device and the RF treatment device are provided as a single device like the fourth embodiment. When only one of the examination operation and the treatment operation is performed, or when the different kinds of tip modules or the different structures of the driver are used in the examination and the treatment, it may be advantageous that the handpieces are separately provided according to the purposes of use like this embodiment. In this case, the examination handpiece 1200 may include invasive-type electrodes like those of the second embodiment, or may include contact-type electrodes like those of the third embodiment.

Here, the controller, the RF generator and the like elements of the main body are electrically/signally connected to the examination handpiece when a user selects the examination mode through the setup unit, and electrically/signally connected to the treatment handpiece when the treatment mode is selected, thereby carrying out the examination and the treatment, respectively.

Embodiments of the disclosure have been described above in detail, but the disclosure is not limited to these embodiments. it will be appreciated by a person having ordinary skill in the art to which the disclosure pertains that various changes or modifications can be made in these embodiments without departing from the scope of technical features of the disclosure defined in the appended claims.

1. A radio frequency (RF) treatment device comprising:
an RF generator configured to generate RF energy;
a plurality of RF electrodes connected to the RF generator through an RF circuit, selectively inserted in body tissue, and configured to transfer the RF energy to the body tissue;
a sensor configured to detect a loss of RF energy transferred to the body tissue due to impedance characteristics of the body tissue;
an impedance adjuster provided on the RF circuit and configured to have variable impedance; and
a controller configured to control the impedance adjuster to reduce a loss of RF energy transferred to the body tissue, based on information detected by the sensor.

2. The RF treatment device of claim 1, wherein the RF electrode is inserted up to a fat layer in a body and configured to transfer RF energy, and the sensor is configured to detect a loss of RF energy due to impedance characteristics of the fat layer.

3. The RF treatment device of claim 1, wherein the sensor is configured to measure power supplied from the RF generator, and voltage and current applied through an RF electrode, and detect a loss of RF energy due to the impedance characteristics of the tissue.

4. The RF treatment device of claim 3, wherein the sensor is configured to detect a loss of the RF energy based on power of the RF energy generated by the RF generator and power of RF energy calculated with the measured voltage and current.

5. The RF treatment device of claim 3, wherein the controller controls the impedance adjuster to increase RF energy to be transferred to the tissue or decrease a phase difference between the measured current and voltage.

6. The RF treatment device of claim 5, wherein the impedance adjuster comprises a variable capacitor connected in series to the RF circuit.

7. The RF treatment device of claim 6, wherein the variable capacitor is adjusted to reduce a loss of RF energy, after identifying change in the loss of the RF energy through the sensor while controlling capacitance of the variable capacitor to be increased or decreased.

8. The RF treatment device of claim 1, wherein
the controller performs control to carry out a first treatment mode where the RF electrode is inserted in a dermal layer and transfers RF energy, or a second treatment mode where the RF electrode is inserted in a fat layer and transfers RF energy, based on a user's settings, and
the impedance adjuster is controlled to operate in the second treatment mode.

9. The RF treatment device of claim 1, wherein
the controller performs control to carry out an adjustment mode where RF energy is transferred to the body tissue to adjust the impedance adjuster, and a treatment mode where the RF energy is transferred to the body tissue using the adjusted impedance adjuster, and
RF energy provided through the RF generator in the adjustment mode is controlled to be smaller than RF energy provided in the treatment mode.

10. A control method based on a radio frequency (RF) treatment device, comprising:
inserting an RF electrode into body tissue;
transferring RF energy, which is provided from an RF generator to the RF electrode along an RF circuit, to the body tissue;
detecting a loss of RF energy due to impedance characteristics of the body tissue while the RF energy is being transferred to the body tissue;
adjusting impedance of an impedance adjuster provided on the RF circuit to reduce the loss of the RF energy; and
transferring the RF energy to the body tissue by providing the RF energy to the RF electrode through the RF circuit of which the impedance is adjusted.

11. The control method of claim 10, wherein the insertion of the RF electrode comprises inserting an end portion of the RF electrode into a fat layer in a body, and the detection of the loss of the RF energy comprises detecting a loss of RF energy due to impedance characteristics of the fat layer.

12. The control method of claim 10, wherein the detection of the loss of the RF energy comprises detecting a loss of the RF energy by measuring power supplied from the RF generator and voltage and current applied through the RF electrode.

13. The control method of claim 12, wherein the detection of the loss of the RF energy comprises detecting a loss of the RF energy based on comparison between power supplied from the RF generator and power obtained by calculating the measured voltage and current.

14. The control method of claim 12, wherein the adjustment of the impedance of the RF circuit comprises controlling the impedance adjuster to increase the RF energy transferred to the tissue, or decrease a phase difference between the measured current and voltage.

15. The control method of claim 14, wherein the impedance adjuster comprises a variable capacitor connected in series to the RF circuit.

16. The control method of claim 15, wherein the adjustment of the impedance of the RF circuit comprises a first stage where the loss of the RF energy is detected by adjusting impedance to increase capacitance of the variable capacitor; a second stage where the loss of the RF energy is detected by adjusting the impedance to decrease the capacitance of the variable capacitor, and a third stage where the capacitance of the variable capacitor is adjusted to reduce the loss of the RF energy based on detection results of the first stage and the second stage.

17. The control method of claim 10, further comprising setting a treatment mode between a first treatment mode where the RF electrode transfers RF energy as inserted in a dermal layer and a second treatment mode where the RF electrode transfers RF energy as inserted in a fat layer,
wherein, when the second treatment mode is selected, the adjustment of the impedance of the RF circuit is performed.

18. A treatment method based on a radio frequency (RF) treatment device, comprising:
inserting an RF electrode into a fat layer;
transferring RF energy, which is provided from an RF generator to the RF electrode along an RF circuit, to the fat layer;
detecting a loss of RF energy due to capacitance characteristics of the fat layer while the RF energy is being transferred to the fat layer;
adjusting an impedance adjuster provided on the RF circuit to reduce the loss of the RF energy transferred to the fat layer; and
treating the body tissue by providing the RF energy to the RF electrode through the RF circuit of which the impedance is adjusted.

19. The treatment method of claim 18, wherein the fat layer is treated for at least one of removal of the fat layer, microcontouring-plasty, and skin elasticity improvement.
